(19) United States
(12) Patent Application Publication
(10) Pub. No.: US 2010/0322912 A1
(43) Pub. Date: Dec. 23, 2010

(54) Combination Enzyme Therapy for Gastric Digestion of Dietary Gluten in Celiac Sprue Patients

(76) Inventors: Chaitan Khosla, Palo Alto, CA (US); Jennifer Ehren, San Diego, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/746,495

(22) PCT Filed: Dec. 8, 2008
(86) PCT No.: PCT/US2008/013492

§ 371 Date: Jul. 28, 2010

Related U.S. Application Data
(60) Provisional application No. 61/007,113, filed on Dec. 10, 2007.

(51) Int. Cl. A61K 38/54; A61P 1/14

(52) U.S. Cl. 424/94.2

(57) ABSTRACT

Combination enzyme products and methods of use thereof are provided. Aspergillopepsin I is combined with a protease enzyme that provides for an additive or synergistic effect in the digestion of toxic gluten oligopeptides. The enzyme products are useful in the treatment of Celiac Sprue patients, particularly for patients who continue to exhibit signs or symptoms of active disease despite following a gluten-free diet.

### BACKGROUND OF THE INVENTION

In 1953, it was first recognized that ingestion of gluten, a common dietary protein present in wheat, barley and rye causes disease in certain sensitive individuals. Gluten is a complex mixture of glutamine- and proline-rich glutenin and prolamine molecules, which is thought to be responsible for disease induction. Ingestion of such proteins by sensitive individuals produces flattening of the normally luxurious, rug-like epithelial lining of the small intestine known to be responsible for efficient and extensive terminal digestion of peptides and other nutrients. Clinical symptoms of Celiac Sprue include fatigue, chronic diarrhea, malabsorption of nutrients, weight loss, abdominal distension, anemia, as well as a substantially enhanced risk for the development of osteoporosis and intestinal malignancies (lymphoma and carcinoma). The disease has an incidence of approximately 1 in 200 in European populations.

A related disease is dermatitis herpetiformis, which is a chronic eruption characterized by clusters of intensely pruritic vesicles, papules, and urticaria-like lesions. IgA deposits occur in almost all normal-appearing and perilesional skin. Asymptomatic gluten-sensitive enteropathy is found in 75 to 90% of patients and in some of their relatives. Onset is usually gradual. Itching and burning are severe, and scratching often obscures the primary lesions with eczematization of nearby skin, leading to an erroneous diagnosis of eczema. Strict adherence to a gluten-free diet for prolonged periods may control the disease in some patients, obviating or reducing the requirement for drug therapy. Dapsone, sulfapyridine and colchicines are sometimes prescribed to relieve itching.

Celiac Sprue is generally considered to be an autoimmune disease and the antibodies found in the serum of Celiac patients support a theory of an immunological nature of the disease. Antibodies to tissue transglutaminase (tTG) and gliadin appear in almost 100% of the patients with active Celiac Sprue, and the presence of such antibodies, particularly of the IgA class, has been used in detection of the disease.

The large majority of patients express the HLA-DQ2 [DQ (a1*0501, b1*02)] and/or DQ8 [DQ (a1*0301, b1*0302)] molecules. It is believed that intestinal damage is caused by interactions between specific gliadin oligopeptides and the HLA-DQ2 or DQ8 antigen, which in turn induce proliferation of T lymphocytes in the sub-epithelial layers. T helper 1 cells and cytokines apparently play a major role in a local inflammatory process leading to villus atrophy of the small intestine.

At the present time there is no good therapy for the disease, except to completely avoid all foods containing gluten. Although gluten withdrawal has transformed the prognosis for children and substantially improved it for adults, some people still die of the disease, mainly adults who had severe disease at the outset. A prevalent cause of death is lymphoreticular disease (especially intestinal lymphoma). It is not known whether a gluten-free diet diminishes this risk. Apparent clinical remission is often associated with histologic relapse that is detected only by review biopsies or by increased EMA titers.

Gluten is so widely used, for example in commercial soups, sauces, ice creams, hot dogs, and other foods, that patients need detailed lists of foodstuffs to avoid and expert advice from a dietitian familiar with celiac disease. Ingesting even small amounts of gluten may prevent remission or induce relapse. Supplementary vitamins, minerals, and hematinics may also be required, depending on deficiency. A few patients respond poorly or not at all to gluten withdrawal, either because the diagnosis is incorrect or because the disease is refractory. In the latter case, oral corticosteroids (e.g., prednisone 10 to 20 mg bid) may induce response.

There are currently no approved drugs or medical foods for patients with clinically diagnosed celiac sprue who still exhibit signs or symptoms of active disease despite following a gluten-free diet. Maintaining a completely gluten-free diet is very challenging. Even highly motivated patients who diligently strive to maintain a strict dietary regimen will be affected due to inadvertent or background exposure to gluten. Total exclusion of dietary gluten is virtually impossible to maintain, because gluten is one of the most common food ingredients, perhaps second only to sugar. Moreover, gluten is an unlabeled ingredient in most packaged, bottled, and canned foods in the United States. Gluten-free groceries are also significantly more expensive (in many cases, greater than two-fold) than equivalent gluten-containing products. Perhaps not surprisingly, as many as 70% of patients with celiac sprue who are in clinical remission and who are making an earnest effort to follow a gluten-free diet, have persistent abnormalities in small bowel biopsy specimens. In another study of 22 subjects with celiac sprue in clinical remission who were assessed on two separate occasions 6 weeks apart, 20 subjects (91%) had abnormal fecal fat excretion (a measure of fat absorption) or abnormal urinary xylose excretion (a measure of sugar absorption) on at least one assessment. Inadvertent exposure to gluten has been identified as the leading cause of non-responsive celiac sprue among clinically diagnosed patients who were presumed to be on a gluten-free diet. Therefore, there is an acute need for non-dietary therapies that could ameliorate the exceptional dietary burden on celiac sprue patients and the serious health consequences of inadequately treated disease. The products described herein present a solution to this problem.

### SUMMARY OF THE INVENTION

The present invention provides compositions and methods for treating the symptoms of Celiac Sprue and/or dermatitis herpetiformis by decreasing the levels of toxic gluten oligopeptides in the patient. The present invention builds upon the discovery that presence of certain gluten oligopeptides resistant to cleavage by gastric and pancreatic enzymes results in toxic effects in sensitive individuals and that enzymatic treatment can remove such peptides and their toxic effects.

Combination enzyme products of the invention contain aspergillopepsin (ASP from Aspergillus niger) in combination with a protease enzyme that provides for an additive or synergistic effect in the digestion of toxic gluten oligopeptides. The enzyme products are useful in the treatment of celiac sprue patients, particularly for patients who continue to exhibit signs or symptoms of active disease despite following a gluten-free diet. Due to its superior efficacy under gastric conditions, ASP is able to enhance the efficacy of other gluten-detoxifying enzymes. By combining complementary enzymes, the safe threshold of ingested gluten can be raised, thereby ameliorating the burden of a highly restricted diet for celiac sprue patients; or providing relief for patients who exhibit signs of disease on a gluten-free diet.

In one embodiment of the invention, the combination enzyme product contains ASP and dipeptidyl peptidase IV (DPPIV from Aspergillus oryzae). Neither enzyme alone is able to detoxify gluten under simulated gastric conditions. However, when combined, the two enzymes are able to detoxify dietary gluten, showing a synergistic effect. In another embodiment the combination enzyme product contains ASP and a glutamine specific endoprotease, e.g. EP-B2. In another embodiment, the combination enzyme product contains ASP and a prolyl endopeptidase, optionally in combination with EP-B2.

Administration of the combination enzyme product to a patient results in cleavage of toxic gluten oligopeptides are cleaved into fragments, thereby preventing or relieving their toxic effects in Celiac Sprue or dermatitis herpetiformis patients. These enzyme combination products are especially important for patients who continue to exhibit signs or symptoms of active disease despite following a gluten-free diet.

The invention provides compositions and methods for the administration of enteric formulations of these enzymes. In another aspect of the invention, stabilized forms of the enzymes are administered to the patient in which stabilized forms are resistant to digestion in the stomach, e.g. to acidic conditions. In one aspect of the invention, a foodstuff is treated with these enzymes prior to consumption by the patient. In another aspect of the invention, the enzymes are administered to a patient and acts internally to destroy the toxic oligopeptides.

In yet another aspect, the invention provides pharmaceutical formulations containing two or more enzymes and a pharmaceutically acceptable carrier. Such formulations may include formulations in which the enzymes are contained within an enteric coating that allows delivery of the active agent to the intestine and formulations in which the active agents are stabilized to resist digestion in acidic stomach conditions. The formulation may comprise one or more enzymes or a mixture or “cocktail” of agents having different activities.

These and other aspects and embodiments of the invention are described in more detail below.

### BRIEF DESCRIPTION OF THE DRAWINGS

FIG. 1: Digestion of Gluten in Whole Wheat Bread by ASP. The ability of ASP to digest dietary gluten under gastric conditions was tested by incubating 600 mg of whole wheat bread (63.2 mg gluten protein, suspended in dilute acid at a final substrate concentration of ˜14 mg/mL) with ASP (0.56 mg/ml). HPLC traces correspond to residual protein from the whole wheat bread at the end of each assay. Each reaction mixture was incubated in 0.01 N HCl containing 0.6 mg/ml pepsin for 60 min at 37° C. The pH at the start of both reactions was ˜4.5. The internal RP-HPLC standard (N-α-p-Tosyl-L-Arginine methyl ester) elutes ˜16 min. Under these HPLC conditions, most immunotoxic gluten peptides have retention times longer than 12.5 min. For example, representative antigenic gluten oligopeptides comprised of 9, 11, 12, 14, 21 and 28 residues elute at 12.5 min, 18.5 min, 21.5 min, 20 min, 22.5 min and 22 min, respectively. Most undigested gluten (or minimally digested peptides longer than 30 residues) binds tightly to the guard column, and is therefore not visualized in the blue HPLC trace. In contrast, virtually all the protein content of bread is adequately proteolyzed in the presence of ASP so as to be visible in the green HPLC trace.

FIG. 2: Effect of DPPIV (a.k.a. peptidase P) on Gluten Digestion in Whole Wheat Bread. HPLC traces correspond to residual protein from the whole wheat bread after 60 min incubation under simulated gastric conditions with 0.56 mg/ml DPPIV or 0.83 mg/ml ASP+0.56 mg/ml DPPIV. The experimental procedures were identical to those described in the caption to FIG. 1.

FIG. 3: Effect of gastric ASP+DPPIV exposure on Gluten Digestion in Whole Wheat Bread following Simulated Duodenal Treatment. HPLC traces correspond to residual protein from the whole wheat bread after 60 min incubation under simulated gastric conditions, followed by simulated duodenal digestion for 30 min. The duodenal phase of digestion was simulated by adjusting the gastric digest to pH 6 followed by addition of 0.375 mg/ml trypsin and 0.375 mg/ml chymotrypsin. The gastric phase samples were either treated with pepsin alone or 0.83 mg/ml ASP+0.56 mg/ml DPPIV in addition to pepsin. The experimental procedures for simulated gastric digestion were identical to those described in the legend to FIG. 1.

FIG. 4: Antibody A1 competitive ELISA analysis of whole-wheat bread treated under simulated gastric conditions. Each trace corresponds to serial dilutions of residual protein from whole wheat bread that has been treated with pepsin alone, pepsin+0.83 mg/ml ASP, or pepsin+0.83 mg/ml ASP+1.1 mg/ml DPPIV under simulated gastric conditions for 60 min. The three samples were prepared similarly to those samples analyzed by HPLC in FIGS. 1 and 2.

FIG. 5: Effect of ASP on Gluten Digestion by EP-B2 and SC PEP. HPLC traces correspond to residual protein from the whole wheat bread after 60 min incubation under simulated gastric conditions (including pepsin) with vehicle (blue trace), 0.56 mg/ml EP-B2+2 units SC PEP (green trace), or 0.56 mg/ml EP-B2+2 units SC PEP+0.83 mg/ml ASP. The experimental procedures were identical to those described in the caption to FIG. 1.

### DETAILED DESCRIPTION OF THE EMBODIMENTS

The gluten detoxifying activities of enzymes were evaluated individually and in combination, including ASP from Aspergillus niger. As detailed below, it was found that ASP provided a potent detoxification of gluten when combined with a second proteolytic enzyme. Such a combination formulation offers the potential advantage of affordable, near-term supportive therapy for patients where dietary intervention alone is inadequate.

Formulations of the invention include, without limitation, combinations of ASP with glutamine-specific endoprotease, e.g. EP-B2, a cysteine endoprotease from germinating barley seeds; with proline-specific endopeptidase; and/or with DPPIV. A combination of ASP with aspergillus DPPIV at a ratio that provides for additive or synergistic activity is preferred.

These combinations rapidly detoxify gluten under simulated gastrointestinal conditions. The enzymes are formulated at a ratio (on a weight:weight basis) that provides for optimum combined activity, preferably a synergistic combined activity where the gluten detoxification is greater than that found for either enzyme when used as a single detoxifying agent. The ratio of ASP to a second proteolytic enzyme may range from about 100:1, about 20:1, about 10:1, about 5:1, about 2:1, about 1:1, about 1:2, about 1:5, about 1:10, about 1:20, or about 1:100.

The methods of the invention can be used for prophylactic as well as therapeutic purposes. As used herein, the term “treating” refers both to the prevention of disease and the treatment of a disease or pre-existing condition. The invention provides a significant advance in the treatment of ongoing disease by stabilizing or improving the patient's clinical symptoms. Such treatment is desirably performed prior to loss of function in the affected tissues but can also help to restore lost function or prevent further loss of function. Evidence of therapeutic effect may be any diminution in the severity of disease, particularly as measured by the severity of symptoms such as fatigue, chronic diarrhea, malabsorption of nutrients, weight loss, abdominal distension, anemia and other symptoms of Celiac Sprue. Other disease indicia include the presence of antibodies specific for gluten, the presence of antibodies specific for tissue transglutaminase, the presence of pro-inflammatory T cells and cytokines, damage to the villus structure of the small intestine as evidenced by histological or other examination, enhanced intestinal permeability and the like.

Patients that may be treated by the methods of the invention include those diagnosed with Celiac Sprue through one or more of serological tests: anti-gliadin antibodies, anti-transglutaminase antibodies or anti-endomysial antibodies; endoscopic evaluation, e.g. to identify celiac lesions; histological assessment of small intestinal mucosa, e.g. to detect villous atrophy, crypt hyperplasia, or infiltration of intra-epithelial lymphocytes; and any GI symptoms dependent on inclusion of gluten in the diet. Amelioration of the above symptoms upon introduction of a strict gluten-free diet is a key hallmark of the disease. However, analysis of celiac patients has shown that a high level of patients believed to be in remission are, in fact, suffering mal-absorption, as evidenced by indicia including but without limitation to xylose absorption tests, fecal fat analysis, lactulose/mannitol permeability tests, and the like. This invention is especially pertinent to patients who do not respond to a gluten-free diet.

Given the safety of oral proteases, they also find a prophylactic use in high-risk populations, such as Type I diabetics, family members of diagnosed celiac patients, HLA-DQ2 positive individuals, and/or patients with gluten-associated symptoms that have not yet undergone formal diagnosis. Such patients may be treated with regular-dose or low-dose (10-50% of the regular dose) enzyme. Similarly, temporary high-dose use of such an agent is also anticipated for patients recovering from gluten-mediated enteropathy in whom gut function has not yet returned to normal assessed by mean such as fecal fat excretion assays.

Patients that can benefit from the present invention may be of any age and include adults and children. Children in particular benefit from prophylactic treatment since prevention of early exposure to toxic gluten peptides can prevent initial development of the disease. Children suitable for prophylaxis can be identified by genetic testing for predisposition, e.g. by HLA typing; by family history, by T cell assay or by other medical means. As is known in the art, dosages may be adjusted for pediatric use.

Although specific enzymes are exemplified herein, any of a number of alternative enzymes and methods apparent to those of skill in the art upon contemplation of this disclosure are equally applicable and suitable for use in practicing the invention. The methods of the invention, as well as tests to determine their efficacy in a particular application, can be carried out in accordance with the teachings herein using procedures standard in the art. Thus, the practice of the present invention may employ conventional techniques of molecular biology (including recombinant techniques), microbiology, cell biology, biochemistry and immunology within the scope of those of skill in the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

As used herein, the term “glutenase” refers to an enzyme useful in the methods of the present invention that is capable, alone or in combination with endogenously or exogenously added enzymes, of cleaving toxic oligopeptides of gluten proteins of wheat, barley, oats and rye into non-toxic fragments. Gluten is the protein fraction in cereal dough, which can be subdivided into glutenins and prolamines, which are subclassified as gliadins, secalins, hordeins, and avenins from wheat, rye, barley and oat, respectively. For further discussion of gluten proteins, see the review by Wieser (1996) Acta Paediatr Suppl. 412:3-9, incorporated herein by reference.

The terms “protease” or “peptidase” can refer to a glutenase and as used herein describe a protein or fragment thereof with the capability of cleaving peptide bonds, where the scissile peptide bond may either be terminal or internal in oligopeptides or larger proteins. Preferably the enzyme is gastrically active. Each of the proteases described herein can be engineered to improve desired properties such as enhanced specificity toward toxic gliadin sequences, improved tolerance for longer substrates, increased acid stability and/or pepsin resistance, enhanced resistance to proteolysis by the pancreatic enzymes and improved shelf-life. The desired property can be engineered via standard protein engineering methods.

Aspergillopepsin I, (ASP) is a broad spectrum protease belonging to the peptidase A1 family. The genetic sequence is publicly available and may be accessed at GenBank ID Q12567 or BAA08639. The enzyme generally favors hydrolysis of proteins at hydrophobic residues.

Dipeptidylpeptidase IV (DPPIV) is a serine exopeptidase. Examples of DPPIV enzymes include Aspergillus spp. (e.g. Byun et al. (2001) J. Agric. Food Chem. 49, 2061-2063), ruminant bacteria such as Prevotella albensis M384 (NCBI protein database Locus #CAC42932), dental bacteria such as Porphyromonas gingivalis W83 (Kumugai et al. (2000) Infect. Immun. 68, 716-724), lactobacilli such as Lactobacillus helveticus (e.g. Vesanto, et al, (1995) Microbiol. 141, 3067-3075) and Lactococcus lactis (Mayo et al., (1991) Appl. Environ. Microbiol. 57, 38-44). Other DPPIV candidates can readily be recognized based upon homology to the above enzymes. DPPIV from Aspergillus oryzae (GenBank ID CAA0534) is of particular interest. The enzyme is described in detail in U.S. Pat. No. 6,309,868 issued Oct. 30, 2001, herein specifically incorporated by reference. Homologues of these enzymes of the invention may be found in publicly available sequence databases and the methods of the invention include such homologues.

Prolyl endopeptidase, PEP, belongs to the serine protease superfamily of enzymes and have a conserved catalytic triad composed of a Ser, His and Asp residues. Some of these enzymes have been characterized, e.g. the enzymes from F. meningoscepticum, Aeromonas hydrophila, Aeromonas punctata, Novosphingobium capsulatum, Pyrococcus furiosus and from mammalian sources are biochemically characterized PEPs. An enzyme of interest is Sphingomonas capsulata PEP (Genbank ID# AB010298).

Other PEPs of interest include Flavobacterium meningosepticum PEP (Genbank ID # D10980), Myxococcus xanthus PEP (Genbank ID# AF127082), and Aspergillus niger PEP (Genbank ID# AX458699).

Glutamine-specific proteases are also of interest for combination products with ASP such as cysteine endoproteinase EP-B2, Hordeum vulgare endoprotease (Genbank accession U19384) and the like.

Among gluten proteins with potential harmful effect to Celiac Sprue patients are included the storage proteins of wheat, species of which include Triticum aestivum, Triticum aethiopicum, Triticum baeoticum, Triticum militinae, Triticum monococcum, Triticum sinskajae, Triticum timopheevii, Triticum turgidum, Triticum urartu, Triticum vatilovii, Triticum zhukovskyi, etc. A review of the genes encoding wheat storage proteins may be found in Colot (1990) Genet Enq (NY) 12:225-41. Gliadin is the alcohol-soluble protein fraction of wheat gluten. Gliadins are typically rich in glutamine and proline, particularly in the N-terminal part. For example, the first 100 amino acids of α- and γ-gliadins contain ˜35% and ˜20% glutamine and proline residues, respectively. Many wheat gliadins have been characterized, and as there are many strains of wheat and other cereals, it is anticipated that many more sequences will be identified using routine methods of molecular biology.

For the purposes of the present invention, toxic gliadin oligopeptides are peptides derived during normal human digestion of gliadins and related storage proteins described above from dietary cereals, e.g. wheat, rye, barley and the like. Such oligopeptides are believed to act as antigens for T cells in Celiac Sprue. For binding to Class II MHC proteins, immunogenic peptides are usually from about 8 to 20 amino acids in length, more usually from about 10 to 18 amino acids. Such peptides may include PXP motifs, such as the motif PQPQLP. Determination of whether an oligopeptide is immunogenic for a particular patient is readily determined by standard T cell activation and other assays known to those of skill in the art.

The amino acid sequence of a glutenase, e.g. a naturally occurring glutenase, can be altered in various ways known in the art to generate targeted changes in sequence and additional glutenase enzymes useful in the formulations and compositions of the invention. Such variants will typically be functionally-preserved variants, which differ, usually in sequence, from the corresponding native or parent protein but still retain the desired biological activity. Variants also include fragments of a glutenase that retain enzymatic activity. Various methods known in the art can be used to generate targeted changes: e.g. phage display in combination with random and targeted mutations, introduction of scanning mutations and the like.

A variant can be substantially similar to a native sequence, i.e. differing by at least one amino acid, and can differ by at least two but usually not more than about ten amino acids (the number of differences depending on the size of the native sequence). The sequence changes may be substitutions, insertions or deletions. Scanning mutations that systematically introduce alanine or other residues may be used to determine key amino acids. Conservative amino acid substitutions typically include substitutions within the following groups: (glycine, alanine), (valine, isoleucine, leucine), (aspartic acid, glutamic acid), (asparagine, glutamine), (serine, threonine), (lysine, arginine) and (phenylalanine, tyrosine).

Glutenase fragments of interest include fragments of at least about 20 contiguous amino acids—more of at least about 50 contiguous amino acids—but may comprise 100 or more amino acids up to the complete protein or may extend further to comprise additional sequences. In each case, the key criterion is whether the fragment retains the ability to digest the toxic oligopeptides that contribute to the symptoms of Celiac Sprue.

Modifications of interest that do not alter primary sequence include chemical derivatization of proteins such as acetylation or carboxylation. Other modifications included are those of glycosylation: modifying the glycosylation patterns of a protein during its synthesis and processing or in further processing steps or exposing the protein to enzymes that affect glycosylation, such as mammalian glycosylating or deglycosylating enzymes. Also embraced are sequences that have phosphorylated amino acid residues, e.g. phosphotyrosine, phosphoserine or phosphothreonine.

Also useful in the practice of the present invention are proteins that have been modified using molecular biological techniques and/or chemistry so as to: improve their resistance to proteolytic degradation and/or acidic conditions such as those found in the stomach, optimize solubility properties or render them more suitable as a therapeutic agent. For example, the backbone of the peptidase can be cyclized to enhance stability (see Friedler et al. (2000) J. Biol. Chem. 275:23783-23789). Analogues of such proteins include those containing residues other than naturally occurring L-amino acids such as. D-amino acids or non-naturally occurring synthetic amino acids.

The proteases of the present invention may be prepared by in vitro synthesis using conventional methods as known in the art. Various commercial synthetic apparatuses are available, for example, automated synthesizers by Applied Biosystems, Inc., Foster City, Calif.; Beckman and other manufacturers. Using synthesizers, one can readily substitute for the naturally occurring amino acids with one or more unnatural amino acids. The particular sequence and the manner of preparation will be determined by convenience, economics, purity required and the like. If desired, various groups can be introduced into the protein during synthesis that allow for linking to other molecules or to a surface. For example, cysteines can be used to make thioethers, histidines can be used for linking to a metal ion complex, carboxyl groups can be used for forming amides or esters, amino groups can be used for forming amides and the like.

The proteases useful in the practice of the present invention may also be isolated and purified in accordance with conventional methods from recombinant production systems and from natural sources, or commercially available sources may be used.

Protease production can be achieved using established host-vector systems in organisms such as E. coli, S. cerevisiae, P. pastoris, Lactobacilli, Bacilli and Aspergilli. Integrative or self-replicative vectors may be used for this purpose. In some of these hosts, the protease is expressed as an intracellular protein and subsequently purified, whereas in other hosts the enzyme is secreted into the extracellular medium. Purification of the protein can be performed by a combination of ion exchange chromatography, Ni-affinity chromatography (or some alternative chromatographic procedure), hydrophobic interaction chromatography and/or other purification techniques. Typically, the compositions used in the practice of the invention will comprise at least 20% by weight of the desired product, more usually at least about 75% by weight, preferably at least about 95% by weight and for therapeutic purposes, usually at least about 99.5% by weight, in relation to contaminants related to the method of preparation of the product and its purification. Usually, the percentages will be based upon total protein.

A Celiac Sprue patient, in addition to being provided with proteases, can be provided an inhibitor of tissue transglutaminase, an anti-inflammatory agent, an anti-ulcer agent, a mast cell-stabilizing agents and/or an anti-allergy agent. Examples of such agents include HMG-CoA reductase inhibitors with anti-inflammatory properties such as compactin, lovastatin, simvastatin, pravastatin and atorvastatin; anti-allergic histamine H1 receptor antagonists such as acrivastine, cetirizine, desloratadine, ebastine, fexofenadine, levocetirizine, loratadine and mizolastine; leukotriene receptor antagonists such as montelukast and zafirlukast; COX2 inhibitors such as celecoxib and rofecoxib; p38 MAP kinase inhibitors such as BIRB-796; and mast cell stabilizing agents such as sodium chromoglycate (chromolyn), pemirolast, proxicromil, repirinast, doxantrazole, amlexanox nedocromil and probicromil.

As used herein, compounds which are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bio-Cat, Inc (Troy, Va.), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Il.), Riedel de Haen A G (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

Compounds useful for co-administration with the proteases can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include: “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992 and J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through online databases (the American Chemical Society, Washington, D.C., www.acs.org may be contacted for more details). Chemicals that are known but not commercially available in catalogs may be prepared by custom chemical synthesis houses where many of the standard chemical supply houses (e.g., those listed above) provide custom synthesis services.

The proteases of the invention and/or the compounds administered therewithin are incorporated into a variety of formulations for therapeutic administration. In one aspect, the agents are formulated into pharmaceutical compositions by combination with appropriate, pharmaceutically acceptable carriers or diluents and are formulated into preparations in solid, semi-solid, liquid or gaseous forms such as tablets, capsules, powders, granules, ointments, solutions, suppositories, injections, inhalants, gels, microspheres and aerosols. As such, administration of the proteases and/or other compounds can be achieved in various ways but usually by oral administration. The proteases and/or other compounds may be systemic after administration or may be localized by virtue of the formulation or by the use of an implant that acts to retain the active dose at the site of implantation.

In pharmaceutical dosage forms, the proteases and/or other compounds may be administered in the form of their pharmaceutically acceptable salts, used alone or in appropriate association or used in combination with other pharmaceutically active compounds. The agents may be combined as previously described to provide a cocktail of activities. The following methods and excipients are exemplary and are not to be construed as limiting the invention.

For oral preparations, the agents can be used alone or in combination with appropriate additives to make tablets, powders, granules or capsules with such conventional additives as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate and if desired, with diluents, buffering agents, moistening agents, preservatives and flavoring agents.

In one embodiment of the invention, the oral formulations comprise enteric coatings so that the active agent is delivered to the intestinal tract. A number of methods are available in the art for the efficient delivery of enterically coated proteins into the small intestinal lumen. Most methods rely upon protein release as a result of the sudden rise of pH when food is released from the stomach into the duodenum or upon the action of pancreatic proteases that are secreted into the duodenum when food enters the small intestine. For intestinal delivery of a PEP and/or a glutamine specific protease, the enzyme is usually lyophilized in the presence of appropriate buffers (e.g. phosphate, histidine, imidazole) and excipients (e.g. cryoprotectants such as sucrose, lactose, trehalose). Lyophilized enzyme cakes are blended with excipients and then filled into capsules enterically coated with a polymeric coating that protects the protein from the acidic environment of the stomach, as well as from the action of pepsin in the stomach. Alternatively, protein microparticles can also be coated with a protective layer. Exemplary films are cellulose acetate phthalate, polyvinyl acetate phthalate, hydroxypropyl methylcellulose phthalate and hydroxypropyl methylcellulose acetate succinate, methacrylate copolymers and cellulose acetate phthalate.

Other enteric formulations comprise engineered polymer microspheres made of biologically erodable polymers which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it—through and between cells. Reference, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71(3):307-18.

Gluten detoxification for a gluten sensitive individual can commence as soon as food enters the stomach since the acidic environment (˜pH 2) of the stomach favors gluten solubilization. Introduction of a protease into the stomach will synergize with the action of pepsin, leading to accelerated destruction of toxic peptides upon entry of gluten into the small intestines of celiac patients. Indeed, since several proteases (including the aforementioned cysteine proteinase from barley) self-activate by cleaving the corresponding pro-proteins under acidic conditions. In one embodiment of the invention, the formulation comprises a pro-enzyme that is activated in the stomach.

In another embodiment, a microorganism, for example bacterial or yeast culture, capable of producing proteases is administered to a patient. Such a culture may be formulated as an enteric capsule; for example, see U.S. Pat. No. 6,008,027, incorporated herein by reference. Alternatively, microorganisms stable to stomach acidity can be administered in a capsule or admixed with food preparations.

Formulations are typically provided in a unit dosage form, where the term “unit dosage form,” refers to physically discrete units suitable as unitary dosages for human subjects. Each unit contains a predetermined quantity of protease in an amount calculated sufficient to produce the desired effect in association with a pharmaceutically acceptable diluent, carrier or vehicle. The specifications for the unit dosage forms of the present invention depend on the particular complex employed and the effect to be achieved, as well as the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, without biologically or otherwise undesirable effects. These salts are prepared from addition of an inorganic base or an organic base to the free acid. Salts derived from inorganic bases include, but are not limited to: sodium, potassium, lithium, ammonium, calcium, magnesium, iron, zinc, copper, manganese, aluminum salts and the like. Preferred inorganic salts are ammonium, sodium, potassium, calcium and magnesium salts. Salts derived from organic bases include, but are not limited to, salts of primary, secondary and tertiary amines; substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the protease may be administered in dosages of 0.01 mg to 1000 mg/kg body weight per day, such as from 1-100 mg/kg/day; for example, 10-100 mg/kg/day for an average person. Efficient proteolysis of gluten in vivo for an adult may require at least about 0.5 units of a therapeutically efficacious enzyme, usually at least about 5 units and more usually at least about 2000 units but not more than about 100,000 units and usually not more than about 10,000,000 units. An effective dose may vary widely depending on the disease, its severity, the age and relative health of the patient being treated, the potency of the compound(s) and other factors. It will be understood by those of skill in the art that the dose can be raised, but that additional benefits may not be obtained by exceeding the useful dosage. Dosages will be appropriately adjusted for pediatric formulation. In children the effective dose may be lower, for example at least about 0.1 mg, or 0.5 mg. In combination therapy involving, for example, an ASP+DPPIV or ASP+EP-B2, a comparable dose of the two enzymes may be given; however, the ratio will be influenced by the relative stability of the two enzymes toward gastric and duodenal inactivation.

Therapeutically effective amount as used herein refers to the amount of active compound or agent that elicits the biological or medicinal response or effect in a cell, tissue, system, animal or human that is being sought, which includes preventing, inhibiting or ameliorating the disease. Enzyme treatment of Celiac Sprue is expected to be most efficacious when administered before or with meals.

Those of skill will readily appreciate that dose levels can vary as a function of the specific enzyme, the severity of the symptoms and the susceptibility of the subject to side effects. Preferred dosages for a given enzyme are readily determinable by those of skill in the art by a variety of means. A preferred means is to measure the physiological potency of a given compound.

Other formulations of interest include formulations of DNA encoding proteases of interest, so as to target intestinal cells for genetic modification. For example, see U.S. Pat. No. 6,258,789, herein incorporated by reference, which discloses the genetic alteration of intestinal epithelial cells.

The therapeutic effect can be measured in terms of clinical outcome or can be determined by immunological or biochemical tests. Suppression of the deleterious T-cell activity can be measured by enumeration of reactive Thi cells, by quantifying the release of cytokines at the sites of lesions or using other assays for the presence of autoimmune T cells known in the art. Alternatively, one can look for a reduction in symptoms of a disease.

Various methods for administration may be employed, preferably using oral administration, for example with meals. The dosage of the therapeutic formulation will vary widely, depending upon the nature of the disease, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, with meals, semi-weekly or otherwise as needed to maintain an effective dosage level.

### EXAMPLES

The following examples are put forth so as to provide those of ordinary skill in the art with a complete disclosure and description of how to make and use the present invention, and are not intended to limit the scope of the invention or to represent that the experiments below are all or the only experiments performed. Efforts have been made to ensure accuracy with respect to numbers used (e.g., amounts, temperature and the like), but some experimental errors and deviations may be present. Unless indicated otherwise, parts are parts by weight, molecular weight is weight average molecular weight, temperature is in degrees Centigrade and pressure is at or near atmospheric.

### Example 1

We sought to evaluate the gluten detoxifying activities of enzymatic ingredients used in commercial dietary supplements, both individually and in combination. Two such enzymes were identified. As detailed below, a combination of these two enzymes offers the potential advantage of affordable, near-term supportive therapy for patients where dietary intervention alone is inadequate.

The salient characteristics of the two enzymes are summarized in Table 1. ASP is from Aspergillus niger and DPPIV is from Aspergillus oryzae. Both enzymes were supplied in powder form by Bio-Cat, Inc (Troy, Va.).

TABLE 1
Specific Activities of ASP and DPP IV. Both enzyme powders were
procured from Bio-Cat, Inc. Protein concentration in each enzyme
powder was measured by a standard Bradford assay using bovine
serum albumin as a reference. Enzymatic activity of ASP was
measured by the HUT (Hemoglobin Units on a Tyrosine basis) assay.
One HUT unit of proteolytic activity is defined as the amount of enzyme
that produces at pH 2.0 and 37 C a change in absorbance at 280 nm
of 0.001 per minute measured as TCA-soluble products of hemoglobin.
Enzymatic activity of DPPIV was measured by UV-Vis
spectrophotometry at 410 nm (A410) using Gly-Pro-paranitroanilide
(Gly-Pro-pNA) as a chromogenic substrate. One unit of DPP IV
activity is defined as the amount of enzyme that produces 1 μmol
of p-nitroaniline per minute at the conditions of pH 4.5 and room
temperature.

| Protease | Protein conc. | Specific Activity | | Assay |
|---|---|---|---|---|
| ASP | 250 mg/g | 18,225 | units/mg | HUT assay |
| DPP IV | 190 mg/g | 2.1 | units/g | Gly-Pro-pNA assay |

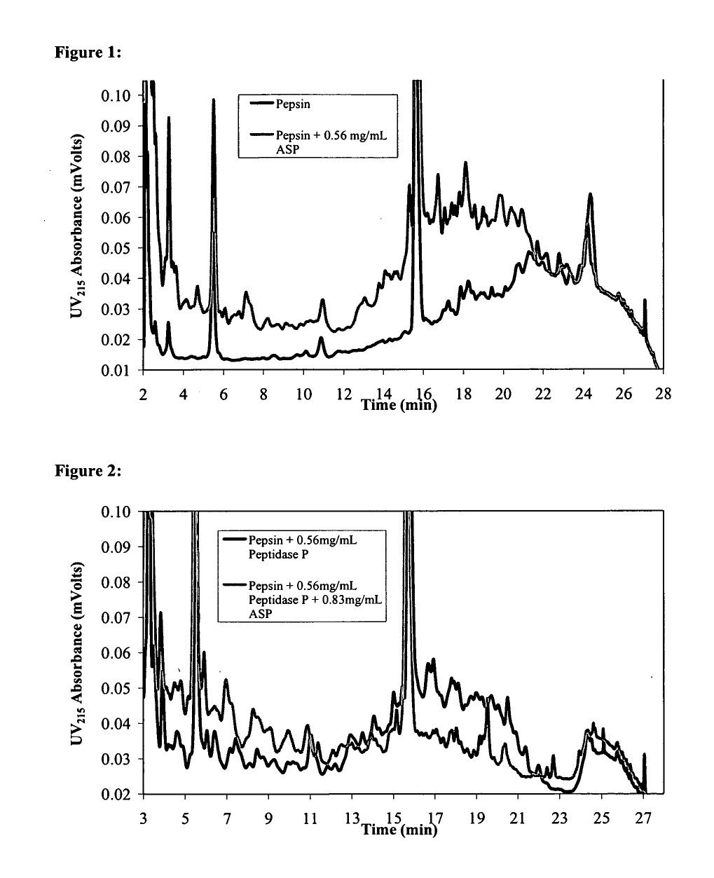

The methods used to analyze the pharmacological efficacy of ASP and DPPIV are described in detail in earlier publications (for example, see Gass (2007) and references therein). These methods are herein incorporated by reference. As shown in FIG. 1, ASP alone enhances the ability of pepsin to proteolyze gluten in whole wheat bread under simulated gastric conditions. In the presence of pepsin alone, only a small fraction of the gluten-derived protein is resolved in the HPLC trace, most of which elutes at retention times longer than 15 min. Co-treatment of bread with pepsin and ASP resulted in extensive gluten proteolysis, causing a significant increase in gluten-derived oligopeptides appearing in the 15-23 min range as well as short (non-toxic) peptides eluting earlier than 10 min.

Although ASP can extensively hydrolyze dietary gluten under simulated gastric conditions, available literature suggests that it cleaves proteins relatively non-specifically. For example, ASP cleaved ribonuclease A at Tyr-X, Phe-X, His-X, Asn-X, Asp-X, Gln-X and Glu-X bonds (Takahashi, 1997). We therefore sought to evaluate the substrate specificity of ASP toward defined gluten peptides and polypeptides. Incubation with the 28-residue peptide, PFPQPQLPYPQPQLPYPQPQLPYPQPQP, from all-gliadin resulted in poor cleavage. The peptide, VQWPQQQPVPQPHQPF, from γ-gliadin was cleaved by ASP after Q5, P8 and V9 residues. Full-length recombinant all-gliadin protein was cleaved by ASP at a wide range of sites including H—X (X=Q,S,L,A), Q-X (X=Q,V,S), R—X (X=L,V,D,N), A-Y, K-Q, L-X (X=Q,V,P) and S—F bonds. Thus, although ASP is able to cleave gluten into relatively short peptides, we anticipated that detoxification of the resulting product mixture would require a second, complementary enzyme.

### Example 2

To detoxify the product resulting from ASP mediated digestion of gluten we chose the exopeptidase DPPIV, which is also widely used as an ingredient in enzyme dietary supplements. Whereas our earlier studies suggested that DPPIV alone could not detoxify gluten under simulated gastric conditions due to the considerable length of peptides generated by pepsin (Hausch, 2002), the unique ability of ASP to cleave gluten into short peptides may render a combination ASP+DPPIV product a viable clinical candidate. To test this hypothesis, whole wheat bread was exposed to DPPIV alone or ASP+DPPIV under simulated gastric conditions. As shown in FIG. 2 (see also FIG. 1 for comparison), by itself DPPIV is unable to adequately reduce the size of pepsin-digested gluten, as evidenced by the abundance of peptides eluting in the 13-23 min range. However, in conjunction with ASP, DPPIV converts gluten into predominantly short, rapidly eluting and presumably non-toxic peptides. This conclusion is also verified by an independent assay as shown in FIG. 4.

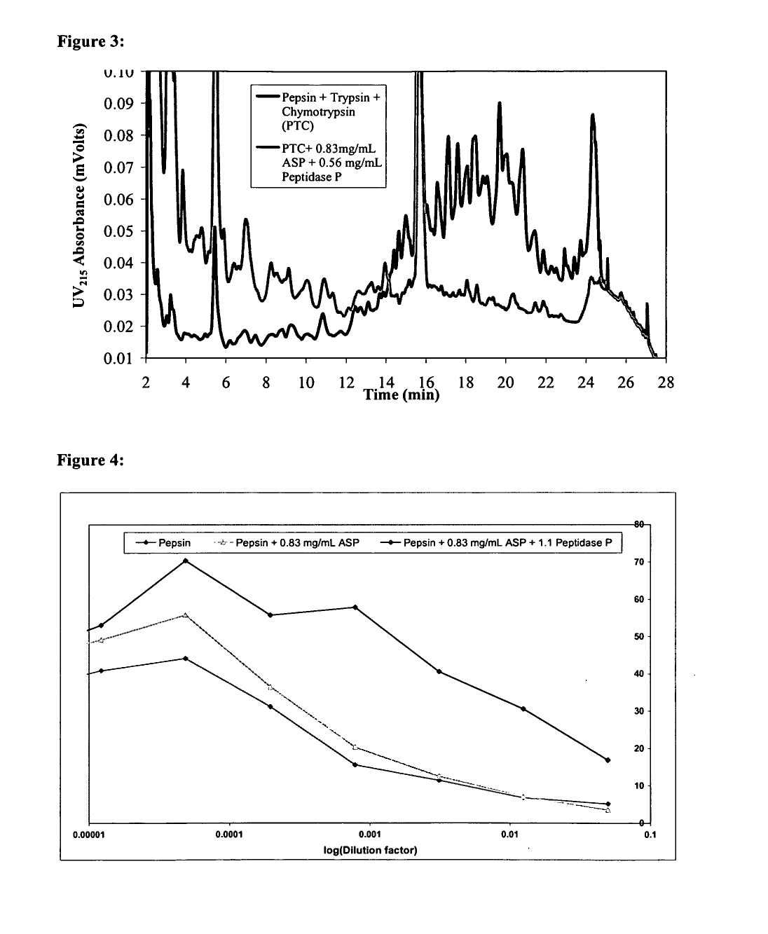

Additional evidence for the complementary or synergistic gluten detoxifying activities of ASP and DPPIV was obtained by treating whole wheat bread with vehicle or ASP+DPPIV under simulated gastric conditions, followed by simulated duodenal conditions. As observed in FIG. 3, extensive exposure of whole wheat bread to simulated gastric conditions (which includes pepsin) followed by simulated duodenal conditions (which includes trypsin and chymotrypsin) results in accumulation of highly immunotoxic oligopeptides eluting between 13-25 min (for example, see Marti, 2005). In contrast, when whole wheat bread is exposed to simulated gastric conditions in the presence of ASP+DPPIV, followed by simulated duodenal conditions, the gluten is thoroughly digested (green trace).

To investigate the dependence of gluten proteolysis on ASP and DPPIV dosing, the ASP and DPPIV concentrations were individually varied between 0.56-1.1 mg/ml (total protein concentration measurements). Assuming that the average volume of contents in the post-prandial stomach is 0.5 L, this corresponds to unit doses of individual enzyme in the range 250-500 mg. The abundance of short (non-toxic) peptides in the 2-12 min range did not change significantly as ASP dose was increased beyond 0.83 mg/ml. In contrast, a modest but measurable increase in short peptides in the 2-12 min range was observed when the DPPIV dose was increased up to 1.1 mg/ml, which was the highest concentration tested. Based on this data, the optimal combination product is anticipated to be a fixed dose ratio product comprised of 250 mg ASP powder and 400 mg DPPIV powder.

To verify the gluten detoxifying activity of this fixed dose ratio product, competitive ELISA measurements were performed on bread treated with ASP+DPPIV, ASP alone, or vehicle. For these experiments, we used a monoclonal antibody, A1, specific for QLPYPQP, a heptapeptide epitope found repeatedly in some of the most proteolytically resistant and immunotoxic gluten peptides (for example, see Shan, 2002). Antibody A1 was a gift from BioMedal S. L., Spain. The ELISA results for whole wheat bread treated with alternative enzyme preparations are shown in FIG. 4. From this data we conclude that ASP+DPPIV reduces the abundance of the A1 antibody epitope by at least 10-fold. Importantly, although ASP alone proteolyzes the gluten in bread extensively, a very modest reduction in abundance of this immunotoxic epitope is observed. This finding reinforces our hypothesis regarding the importance of including DPPIV in an efficacious product for Celiac Sprue. Furthermore, a >10-fold reduction in gluten immunotoxicity also supports our hypothesis that the proposed fixed-dose ratio could increase the safe threshold of dietary gluten up to 1 gram.

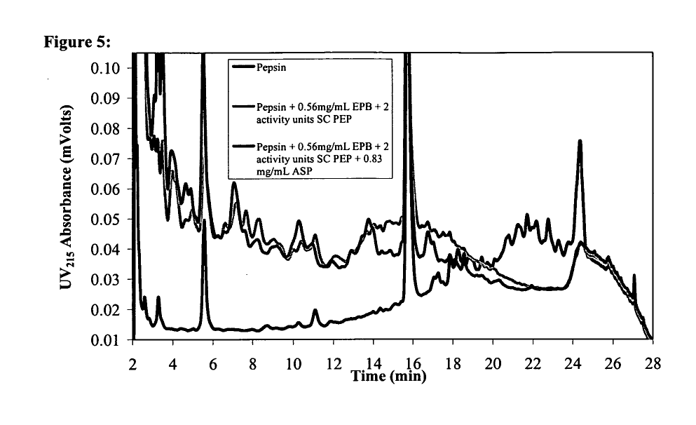

As discussed above, an attractive feature of aspergillopepsin is that, unlike mammalian pepsin, aspergillopepsin is able to extensively hydrolyze dietary gluten into short peptides. This finding suggests that ASP should also be able to complement the glutenase activities of other promising enzymes such as cysteine endoprotease EP-B2 from barley (Bethune, 2006), prolyl endopeptidase AN PEP from A. niger (Stepniak, 2006) and the combination product comprised of EP-B2 and prolyl endopeptidase SC PEP from S. capsulata (Gass, 2007). To test this hypothesis, the activity of ASP was tested in conjunction with EP-B2+SC PEP under simulated gastric conditions. As shown in FIG. 5, the three-enzyme cocktail is able to enhance the extent to which gluten is detoxified as compared to identical concentrations of only EP-B2+SC PEP.

### Materials & Methods

Materials: Whole wheat bread (Alvarado St Sprouted Whole Wheat Bread) was from Alvarado St Bakery (Rohnert Park, Calif.). ASP is from Aspergillus niger and DPPIV is from Aspergillus oryzae. Both enzymes were supplied in powder form by Bio-Cat, Inc (Troy, Va.).

Pepsin was obtained from American Laboratories (Omaha, Nebr.). Trypsin (from bovine pancreas, T4665) and α-chymotrypsin (type II from bovine pancreas, C4129) were from Sigma (St. Louis, Mo.). Substrates for the chromogenic assays for PEP (Suc-Ala-Pro-p-Nitroanilide or Z-Gly-Pro-p-Nitroanilide) and EP-B2 (Z-Phe-Arg-pNA) were from Bachem (Torrance, Calif.).

EP-B2 and SC PEP Enzyme Manufacturing and Testing: EP-B2 was prepared in its zymogen form by Alvine Pharmaceuticals per existing protocol (Vora et al., submitted manuscript). SC PEP was prepared as described previously¹⁵. EP-B2 concentration was between 5.8-15.5 mg/ml in 100 mM Tris-Cl, 5 mM EDTA, 2 mM β-mercaptoethanol, 15% sucrose, pH 8, with specific activity ranging between 800-5000 units/mg. SC PEP was prepared in 20 mM sodium phosphate buffer, pH 7, or phosphate-buffered saline, pH 7.4, at a concentration between 60-90 mg/mL and specific activity of 15-20 units/mg. Enzyme activity assays were performed as described earlier18 (Vora et al., submitted manuscript).

In vitro Whole Wheat Bread Digestion: To evaluate the efficacy of alternative proteases, an in vitro experimental protocol was developed to mimic the ingestion and digestion of whole wheat bread from a grocery store. Alvarado St Sprouted Whole Wheat Bread was selected because of its high protein level (label claim of 4 g protein for 38 g slice). A portion of a bread slice (typically 1 g) was pre-soaked with specified levels of protease solutions formulated in their respective buffers. The bread was divided into 5 or 6 pieces depending upon the experiment.

To initiate the in vitro digestion protocol, the pre-soaked bread pieces were added to a 0.01 N HCl solution (pH 2, pre-incubated at 37° C.) containing 0.6 mg/mL pepsin. Approximately 6.67 mL 0.01 HCl solution was added to 1 g bread (starting weight before any liquid addition) to achieve a final protein concentration of approximately 14 mg/mL in the suspension. The bread pieces were steadily added over 15 min and after addition of each piece, the mixture was manually agitated with a spatula. The pH was approximately 4.5 at the end of the ingestion phase.

The simulated gastric digestion phase was considered to start upon addition of the last bread piece to the 0.01 N HCl solution. The material was incubated at 37° C. for various times (typically, 10 min to mimic short gastric digestion or 60 min to mimic extended gastric digestion). Samples (500 μL) were taken at 0, 10, 30 and 60 min and immediately heated at >95° C. for at least 10 min to inactivate the enzymes. The mixture was manually agitated with a spatula prior to each sampling event.

In experiments where duodenal digestion was simulated, at the end of the gastric phase, the pH was adjusted to 6.0 by the addition of sodium phosphate (15 mg for a 1 g bread digest) and 1 M HCl and/or 1 M NaOH. Pancreatic enzymes (trypsin and chymotrypsin, or trypsin, chymotrypsin, elastase, and carboxypeptidase A), prepared in ˜50 mg/mL stock solutions, were added to yield the following final concentrations: 0.375 mg/mL trypsin, 0.375 mg/mL chymotrypsin, 0.075 mg/mL elastase and 0.075 mg/mL carboxypeptidase A. The final solution was then incubated at 37° C. for up to 30 min. Samples (500-1000 μL) were withdrawn at 10 and 30 min and heat-treated as described above.

Reverse Phase HPLC: Samples from the in vitro whole wheat bread digests were chromatographically separated on a 4.6×150 mm reverse phase C₁₈ protein and peptide column (Grace Vydac, Hesperia, Calif.) using Varian-Rainin Dynamax (Palo Alto, Calif.) SD-200 pumps (1-1.5 ml/min), a Varian 340 UV detector set at 215 nm and a Varian Prostar 430 autosampler. Solvent A was water with 5.0% acetonitrile in water and 0.1% trifluoroacetic acid. Solvent B was 95% acetonitrile in water and 0.1% trifluoroacetic acid. Prior to injection, samples were centrifuged for 10 min at approximately 14,000·g and filtered through a 0.2 μm syringe filter.

### REFERENCES

Abdulkarim A, Burgart L, See J, Murray J (2002) Etiology of nonresponsive celiac disease: results of a systematic approach. Am. J. Gastroenterol. 97: 2016-2021.
Bethune M T, Strop P, Tang Y, Sollid L M, Khosla C (2006) Heterologous expression, purification, refolding and structural-functional characterization of EP-B2, a self-activating barley cysteine endoprotease. Chem. Biol. 13:637-647.
Fasano A, Berti I, Gerarduzzi T, Not T, Collet R B, Drago S, Elitsur Y, Green P H R, Guandalini S, Hill I D, Pietzak M, Ventura A, Thorpe M, Kryszak D, Formaroli F, Wasserman S S, Murray J A, Horvath K (2003) Arch. Intern. Med. 163:286-292.
Gass J, Vora H, Bethune M T, Gray G M, Khosla C (2006) Effect of barley endoprotease EP-B2 on gluten digestion in the intact rat. J. Pharmacol. Exp. Therap. 318:1178-1186.
Gass J, Bethune M T, Siegel M, Khosla C (2007) Combination enzyme therapy for gastric digestion of dietary gluten in celiac sprue patients. Gastroenterol. 133:472-480.
Hausch F, Shan L, Santiago N A, Gray G M, Khosla C (2002) Intestinal digestive resistance of immunodominant gliadin peptides. Am. J. Physiol. Gastrointest. Liver Physiol. 283: G996-G1003.
Holtmeier W, Caspary W F (2006) Celiac disease. Orphanet J. Rare Diseases 1:3-10
Kagnoff M F (2007) Celiac disease: pathogenesis of a model immunogenetic disease. J. Clin. Invest. 117: 41-49.
Lee S K, Lo W, Memeo L, Rotterdam H, Green P H R (2003) Duodenal histology in patients with celiac disease after treatment with a gluten-free diet. Gastrointest Endoscopy 57:187-191.
Leffler D A, Dennis M, Hyett B, Kelly E, Schuppan D, Kelly C P (2007) Etiologies and predictors of diagnosis in nonresponsive celiac disease. Clin. Gastroenterol. Hepatol. 5:445-450.
Marti T, Molberg O, Li Q, Gray G M, Khosla C, Sollid L M (2005) Prolyl endopeptidase-mediated destruction of T cell epitopes in whole gluten: Chemical and immunological characterization. J. Pharmacol. Exp. Therap. 312:19-26.
Piper J L, Gray G M, Khosla C (2004) Effect of prolyl endopeptidase on digestive-resistant gliadin peptides in vivo. J. Pharmacol. Exp. Therap. 311:213-219.
Pyle G G, Paaso B, Anderson B E, Allen D D, Marti T, Li Q, Siegel M, Khosla C, Gray G M (2005b) Effect of pretreatment of food gluten with prolyl endopeptidase on gluten-induced malabsorption in celiac sprue Clin. Gastroenterol. Hepatol. 3:687-694.
Shan L, Molberg O, Parrot I, Hausch F, Filiz F, Gray G M, Sollid L M, Khosla C (2002) Structural basis for gluten intolerance in celiac sprue. Science 297:2275-2279.
Shan L, Qiao S W, Arentz-Hansen H, Molberg O, Gray G M, Sollid L M, Khosla C (2005b) Identification and analysis of multivalent proteolytically resistant peptides from gluten: Implications for celiac sprue. J. Proteome Res. 4:1732-1741.
Siegel M, Bethune M T, Gass J, Ehren J, Xia J, Johannsen A, Stuge T B, Gray G M, Lee P P, Khosla C (1006) Rational design of combination enzyme therapy for celiac sprue. Chem. Biol. 13:649-658.
Stepniak D, Spaenij-Dekking L, Mitea C, Moester M, de Ru A, Baak-Pablo R, van Peelen B, Edens L, Koning F (2006) Highly efficient gluten degradation with a newly identified prolyl endoprotease: implications for celiac disease. Am J Physiol Gastrointest Liver Physiol 291:G621-G629.
Takahashi, K. (1997) The specificity of peptide bond cleavage of acid proteinase A from Aspergillus niger var. macrosporus toward oxidized ribonuclease A. Biosci. Biotechnol. Biochem. 61:381-383.
Talley N J, Valdovinos M, Petterson T M, Carpenter H A, Melton L J 3^{rd} (1994) Epidemiology of celiac sprue: a community-based study. Am. J. Gastroenterol. 89:843-846.

What is claimed is:

1. An enzyme composition:
comprising a therapeutically effective dose of aspergillopepsin I and a second proteolytically active enzyme, wherein the combination of enzymes provides increased detoxification of gluten peptides relative to aspergillopepsin alone.

2. The enzyme composition of claim 1, wherein the aspergillopepsin I is Aspergillus niger aspergillopepsin I.

3. The enzyme composition of claim 1, wherein the second proteolytically active enzyme is DPPIV.

4. The enzyme composition of claim 3, wherein the DPPIV is Aspergillus oryzae DPP IV.

5. The enzyme composition of claim 4, wherein the ratio of enzymes is about 1:1 by weight of active enzyme.

6. The enzyme composition of claim 2, wherein the ratio of enzymes is from about 10:1 to about 1:10 by weight of active enzyme.

7. The enzyme composition of claim 2, wherein the ratio of enzymes is from about 100:1 to about 1:100 by weight of active enzyme.

8. The enzyme composition of claim 1, wherein the combination of enzymes provides for a synergistic effect in detoxification of gluten.

9. The enzyme composition of claim 1, wherein the second proteolytically active enzyme is EP-B2.

10. The enzyme composition of claim 1, wherein the second proteolytically active enzyme is SC PEP.

11. The enzyme composition of claim 10, further comprising EP-B2.

12. The enzyme composition of claim 1, wherein the second proteolytically active enzyme is a prolyl endopeptidase.

13. The enzyme composition of claim 1, wherein the second proteolytically active enzyme is A. niger proline endoprotease.

14. A pharmaceutical formulation of the composition set forth in claim 1, further comprising a pharmaceutically acceptable excipient.

15. A method of treating a patient diagnosed with Celiac Sprue by administering an effective dose of pharmaceutical composition of claim 14.

16. The method of claim 15, wherein the patient has an inadequate response to a gluten-free diet.

17. A method of treating a patient diagnosed with dermatitis herpetiformis by administering an effective dose of pharmaceutical composition of claim 14.

18. The method of claim 17, wherein the patient has an inadequate response to a gluten-free diet.
